(19) United States
(12) Patent Application Publication
(10) Pub. No.: US 2018/0059103 A1
(43) Pub. Date: Mar. 1, 2018

(54) DEVICES AND METHODS FOR DETECTION OF BIOMARKERS

(71) Applicant: POC MEDICAL SYSTEMS INC, Livermore, CA (US)

(72) Inventors: SANJEEV SAXENA, LIVERMORE, CA (US); LOUIS EUGENE BURTON, SAN MATEO, CA (US); FRANCIS WILLIAM LAU, OAKLAND, CA (US); ANDREA CUPPOLETTI, LIVERMORE, CA (US); RAJINDER KAUR BHATIA, NEWARK, CA (US)

(73) Assignee: POC MEDICAL SYSTEMS INC, Livermore, CA (US)

(21) Appl. No.: 15/797,502

(22) Filed: Oct. 30, 2017

Related U.S. Application Data
(63) Continuation of application No. PCT/US2016/030959, filed on May 5, 2016.

(51) Int. Cl. G01N 33/543; B01L 3/00; G01N 33/574

(52) U.S. Cl. CPC G01N 33/54366; B01L 3/502761; B01L 3/50273; G01N 33/574; G01N 33/57415; B01L 2300/0803; B01L 2300/0864; B01L 2200/0668; B01L 2300/0627; B01L 2400/0409; G01N 33/54313

(57) ABSTRACT

Devices for detecting biomarkers in a sample and methods for detecting such biomarkers are discussed. The biomarkers may be associated with a particular disease such as breast cancer or another health condition. The devices may contain a disc including a plurality of microfluidic channels extending in a radial direction of the disc, the microfluidic channels pre-loaded with a plurality of capture molecules specific to at least one biomarker. The methods may include introducing a fluid sample into at least one microfluidic channel of a disc, rotating the disc, such that the fluid sample flows radially outward through the at least one microfluidic channel to combine with capture molecules specific to at least one biomarker, and detecting a signal from the disc indicative of a presence of the at least one biomarker contained in the fluid sample.

### CROSS REFERENCE TO RELATED APPLICATIONS

This application is a continuation of and claims the benefit of priority to PCT application PCT/US2016/030959 FILED ON May 5, 2016 which is incorporated by reference herein in its entirety.

### TECHNICAL FIELD

The present disclosure generally relates to the detection of biomarkers associated with a health condition, e.g., to assist in medical screening and/or diagnosis.

### BACKGROUND

Biomarkers can be useful indicators of a potential medical condition. Yet, diseases can involve numerous biochemical species and reactions. For example, breast cancer is a complex disease which can have multiple pathways to generate the same stage of disease with similar symptoms for the patient. While researchers have sought new biomarkers, the ability to screen for various diseases remains limited. Cancer antigen 125 (CA 125) and carcinoembryonic antigen (CEA) have been reported as biomarkers for ovarian and colorectal cancer, respectively, whereas a single biomarker has yet to be identified for breast cancer. Research over the past decade has focused on discovering new biomarkers to provide accurate diagnosis of disease, guide therapeutic decision making, and predict future patterns of disease. Yet some diseases like breast cancer may be not a single disease, but a genetically heterogeneous set of diseases. For such conditions, it may be difficult or not possible to diagnose with a single biomarker.

### SUMMARY

The present disclosure includes a device comprising a disc including a plurality of microfluidic channels extending in a radial direction of the disc, the microfluidic channels comprising a plurality of capture molecules specific to at least one biomarker, wherein each capture molecule of the plurality of capture molecules is attached to a particle. The disc may include, for example, from 1 to 100 microfluidic channels, such as from 12 to 60 microfluidic channels. In some aspects, the plurality of microfluidic channels may comprise a first channel including a plurality of first capture molecules specific to a first biomarker and a second channel including a plurality of second capture molecules specific to a second biomarker different from the first biomarker. Further, for example, the plurality of microfluidic channels may include a first microfluidic channel containing a first set of capture molecules specific to a first biomarker, a second microfluidic channel containing a second set of capture molecules specific to a second biomarker different from the first biomarker, and a third microfluidic channel containing a third set of capture molecules specific to a third biomarker different from each of the first biomarker and the second biomarker. Additionally or alternatively, the disc may comprise at least one sample inlet configured to separate a raw sample into one or more components, such as, e.g., separating plasma from whole blood.

According to some aspects of the present disclosure, each capture molecule may be attached to a microbead having an average diameter ranging from 100 nm to 10 μm, such as, e.g., an average diameter of about 1 μm. Each capture molecule of the plurality of capture molecules may comprise, for example, an antibody specific to at least one biomarker. In at least one example, each capture molecule may comprise an antibody specific to only one biomarker. In some aspects, the plurality of capture molecules may include capture molecules specific for biomarkers chosen from human estrogen receptor 2 (Her-2), matrix metallopeptidase-2 (MMP-2), cancer antigen 15-3 (CA 15-3), vascular endothelial growth factor (VEGF), or osteopontin (OPN). For example, the plurality of capture molecules may include capture molecules specific for Her-2, MMP-2, CA 15-3, and OPN. In some aspects, the plurality of capture molecules may include capture molecules specific for biomarkers chosen from human estrogen receptor 2 (Her-2), matrix metallopeptidase-2 (MMP-2), cancer antigen 15-3 (CA 15-3), vascular endothelial growth factor (VEGF), osteopontin (OPN), tumor protein p53 (p53), cancer antigen 125 (CA 125), carcinoembryonic antigen (CEA), or serum estrogen receptor (SER). For example, the plurality of capture molecules may include at least one antibody chosen from antibodies Clone 191924, Clone 36006.211, Clone M8071022, or Clone 190312. The plurality of capture molecules may include at least one capture molecule or multiple capture molecules blocked by a blocking agent.

According to some aspects of the present disclosure, the plurality of microfluidic channels may comprise a plurality of detection molecules, each detection molecule including a detectable label. In some examples, the device also may comprise a power source and/or a detector, e.g., for detection of the at least one biomarker. For example, the detection may be configured to detect fluorescence and/or chemiluminescence.

The present disclosure further includes methods for detection of biomarkers using a device with any combination of features discussed above. The method may comprise, for example, introducing a fluid sample into at least one microfluidic channel of the disc; rotating the disc, such that the fluid sample flows radially outward through the at least one microfluidic channel to combine with at least one capture molecule of the plurality of capture molecules; and detecting a signal from the disc indicative of a presence of at least one biomarker of the fluid sample. The fluid sample may comprise a plurality of biomarkers associated with a health condition. In at least some examples, the health condition may be cancer, e.g., breast cancer.

According to some aspects of the present disclosure, the fluid sample introduced into the microfluidic channel(s) of the disc may comprise at least one biomarker chosen from human estrogen receptor 2 (Her-2), matrix metallopeptidase-2 (MMP-2), cancer antigen 15-3 (CA 15-3), vascular endothelial growth factor (VEGF), osteopontin (OPN), tumor protein p53 (p53), cancer antigen 125 (CA 125), carcinoembryonic antigen (CEA), or serum estrogen receptor (SER). For example, the plurality of capture molecules may include capture molecules specific for biomarkers associated with breast cancer, such as, e.g., capture molecules specific for at least one biomarker chosen from human estrogen receptor 2 (Her-2), matrix metallopeptidase-2 (MMP-2), cancer antigen 15-3 (CA 15-3), vascular endothelial growth factor (VEGF), osteopontin (OPN), tumor protein p53 (p53), cancer antigen 125 (CA 125), carcinoembryonic antigen (CEA), or serum estrogen receptor (SER).

In some aspects, the fluid sample may comprise blood or may be obtained from blood. For example, the fluid sample may comprise human blood, human blood plasma, or human blood serum. In some exemplary methods of the present disclosure, detecting the signal from the disc may include detecting a fluorescence signal of a detection molecule attached to the at least one biomarker in the fluid sample. Additionally or alternatively, detecting the signal from the disc may include detecting chemiluminescence. Further, for example, detecting the signal from the disc may include analyzing the fluid sample with an optical reader to determine a presence or absence of the at least one biomarker in the fluid sample. The presence of the at least one biomarker may be indicative of a medical diagnosis of a disease. For example, the at least one biomarker may include a plurality of biomarkers associated with breast cancer. In some aspects, the method may include comparing a level of each biomarker of the plurality of biomarkers in a first fluid sample obtained from a subject to a level of each biomarker of the plurality of biomarkers in a second fluid sample obtained from a subject. The first and second fluid samples may be obtained from the subject after a period of time has passed, such as one or more weeks or one or more months. For example, the first fluid sample may be obtained from the subject at least one week before the second fluid sample is obtained from the subject. A difference in the levels of one or more of the biomarkers may be indicative of a medical diagnosis of a disease and/or of progression of the disease over time.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments. Any features of an embodiment described herein (e.g., composition, medical device, method of treatment, etc.) may be combined with any other embodiment, and are encompassed by the present disclosure.

FIG. 1 shows an exemplary microfluidic disc, in accordance with some aspects of the present disclosure.

FIG. 2 shows an exemplary microfluidic disc, in accordance with some aspects of the present disclosure.

FIG. 3 is a schematic that illustrates capture molecules attached to a microbead, in accordance with some aspects of the present disclosure.

FIG. 4 is a schematic of an exemplary assay using a microfluidic disc, in accordance with some aspects of the present disclosure.

FIG. 5 shows exemplary components of a device, in accordance with some aspects of the present disclosure.

FIG. 6 shows an exemplary container of a device, in accordance with some aspects of the present disclosure.

FIG. 7 is a graph showing fluorescence measurements for cancer antigen 15-3 (CA 15-3).

FIG. 8 is a graph showing fluorescence measurements for matrix metallopeptidase-2 (MMP-2).

FIG. 9 is a graph showing fluorescence measurements for human estrogen receptor 2 (Her-2).

FIG. 10 is a graph showing fluorescence measurements for osteopontin (OPN).

FIG. 11 is a graph showing fluorescence measurements for vascular endothelial growth factor (VEGF).

### DETAILED DESCRIPTION

Embodiments of the present disclosure may address a need for alternative screening and diagnostic options by providing multiple, mutually complementary biomarkers for a sensitive diagnostic assay. Aspects of the present disclosure may offer certain advantages in screening patients, including large populations, for breast cancer and other diseases and health conditions.

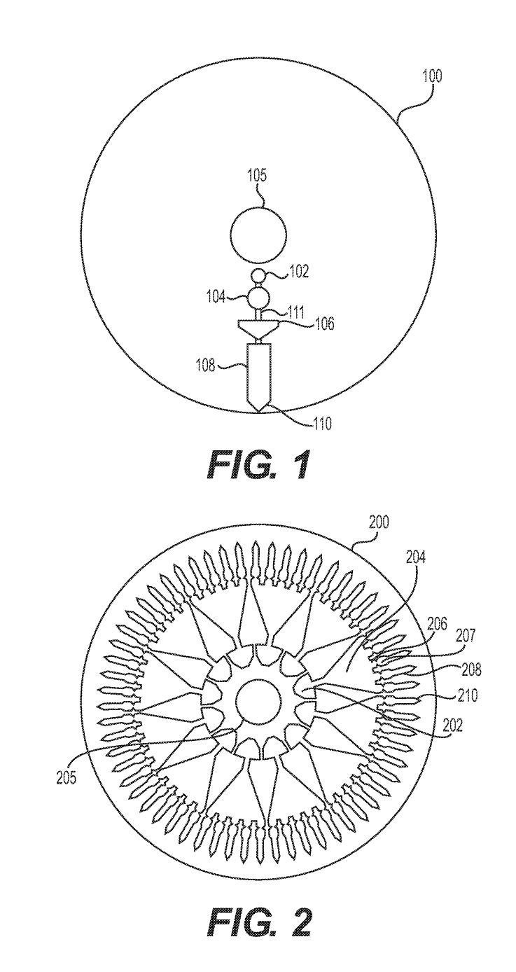

The present disclosure includes using a panel of multiple biomarkers, e.g., to increase the probability to detect the onset of various health conditions. Testing a panel of two or more biomarkers simultaneously may provide a combined effect with a predictive value greater than that provided by a single biomarker. The panels of biomarkers herein may be tested with a suitable analysis platform. In some cases, only a small amount of sample from a patient may be needed, such as, e.g., blood, plasma, or other suitable fluid or tissue sample. Examples of suitable devices may include, but are not limited to, microfluidic devices, such as the Pandora CDx device (POC Medical Systems) and other microfluidic platforms. FIGS. 1 and 2 show exemplary discs comprising microfluidic channels according to some aspects of the present disclosure, and are discussed below. In at least one example, an array for breast cancer comprises multiple biomarkers suitable for analysis on a microfluidic device.

The singular forms “a,” “an,” and “the” include plural reference unless the context dictates otherwise.

The terms “approximately” and “about” refer to being nearly the same as a referenced number or value. As used herein, the terms “approximately” and “about” generally should be understood to encompass ±5% of a specified amount or value.

Aspects of the present disclosure include testing a biological sample from a subject for one or more biomarkers that are associated with a health condition, such as a disease. Further, according to some aspects, data on the presence or absence of various biomarkers, and the amount or level of each biomarker present in the sample, may be analyzed to obtain diagnostic information for the subject. The data may allow for a determination of the probability that the diagnosis is correct.

A sample may be obtained or derived from any subject of interest, including mammalian subjects such as, e.g., human subjects, e.g., patients. Mammalian subjects include both humans and non-humans. Exemplary mammals for which samples may be analyzed according to the methods herein include, but are not limited to, humans, non-human primates, canines, felines, murines, bovines, equines, and porcines.

A sample may comprise blood and/or other liquid samples of biological origin, solid tissue samples such as a biopsy specimen, tissue culture, or cells derived therefrom, and the progeny thereof. A sample may comprise a single cell or more than a single cell, e.g., a plurality of cells. Samples may include clinical samples, cells in culture, cell supernatants, and/or cell lysates. In some aspects, a sample may be of cancerous origin, e.g., obtained from cancerous tissues. For example, the sample may be obtained from cancerous breast tissues.

In some aspects, a sample may be manipulated or processed by one or more procedures or treatment steps after their procurement from a subject. For example, a sample may be treated with one or more reagents, solubilized, and/or enriched for certain components. Enrichment of a sample may include, for example, concentrating one or more constituents of the sample to assist in detection, analysis, and/or identification of those constituent. In at least one example, a sample may be enriched for one or more target proteins and/or polynucleotides prior to exposing the sample to capture molecules for binding and detecting the target(s).

The term “biomarker” generally refers to a chemical or biochemical indicator associated with one or more health conditions. A biomarker may include, but is not limited to, a molecule of interest or a portion of a molecule of interest that is to be detected and/or analyzed. Exemplary biomarkers include, e.g., peptides, proteins, DNA sequences, and RNA sequences. The terms “polypeptide,” “oligopeptide,” “peptide,” and “protein” may be used interchangeably herein to refer to polymers of amino acids of any length that may or may not include chemical modifications. Such a polymer may be linear or branched, may comprise modified amino acids, and/or may be interrupted by non-amino acids. The amino acid polymers may be modified naturally or by intervention. For example, amino acid polymers according to the present disclosure may be modified by disulfide bond formation, glycosylation, lipidation, acetylation, phosphorylation, or any other manipulation or modification, such as conjugation with a labeling component. The amino acid polymers may include polypeptides comprising one or more analogs of an amino acid (including, for example, unnatural amino acids) as well as other chemical/biochemical modifications known in the art.

Biomarkers according to the present disclosure include genetic markers, e.g., DNA sequences of an organism that may be useful in identifying characteristics of that organism. For example, genetic markers associated with a disease or other health condition may include one or more alterations, variations, and/or mutations in a DNA sequence as compared to a DNA sequence that is not associated with the disease or other health condition. A biomarker or combination of biomarkers may be associated with a particular physical condition or health condition, e.g., a disease or disease state. For example, the biomarker(s) may be associated with breast cancer, e.g., late stage breast cancer.

Biomarkers that may be detected and/or analyzed according to the present disclosure include, but are not limited to, human estrogen receptor 2 (Her-2), matrix metallopeptidase-2 (MMP-2), matrix metalloproteinase 9 (MMP-9), cancer antigen 15-3 (CA 15-3), cancer antigen 125 (CA 125), cancer antigen 27.29 (CA 27.29), carcinoembryonic antigen (CEA), vascular endothelial growth factor (VEGF), epidermal growth factor (EGF), hepatocyte growth factor (HGF), tumor specific growth factor (TSGF), tumor specific growth factor (TSGF), osteopontin (OPN), tumor protein p53 (p53), serum estrogen receptor (SER), serum progesterone receptor (SPR), BRCA 1 protein, BRCA 2 protein, prostate specific antigen (PSA), troponin T, troponin I, C-reactive protein (CRP), homocysteine, myoglobin, creatine kinase, adrenocorticotropic hormone (ACTH), alpha-fetoprotein (AFP), anterior gradient 3 (AGR3), apolipoprotein A1 (Apo-A1), D-dimer (DD), dermcidin, high molecular weight kininogen (HMWK), leptin, myeloperoxidase (MPO), macrophage migration inhibitory factor (MIF), mucin-like carcinoma associated antigen (MCA), plasminogen activator inhibitor-1 (PAI-1), prolactin, soluble CD40 ligand (sCD40L), soluble epidermal growth factor receptor (sEGFR), soluble vascular cell adhesion molecule 1 (sVCAM-1), soluble vascular endothelial growth factor receptor 1 (sVEGFR1), soluble vascular endothelial growth factor receptor 2 (sVEGFR2), tissue polypeptide antigen (TPA), thymidylate synthase (TS), urokinase plasminogen activator (uPA), vitamin D-binding protein (VDBP), and vitronectin (VN). These biomarkers can include fragments, splice variants, and/or full length peptides, or any other variations. It is understood that the present disclosure is not limited to the biomarkers listed, and additional biomarkers are encompassed and contemplated for the systems, devices, and methods herein.

The term “capture molecule” generally refers to a molecule that may bind to a target (e.g., a target molecule, such as a biomarker). For example, a capture molecule may have one binding site, or a plurality of two or more binding sites. Capture molecules according to the present disclosure may be capable of binding to only one target (e.g., the capture molecule being specific to one particular target), to a select number of targets (e.g., the capture molecule being specific to two or more targets), or to a plurality of target and non-target species.

In some aspects of the present disclosure, a biomarker may bind to a capture molecule. A molecule or other chemical/biochemical species may be said to exhibit “binding” if it reacts or associates more frequently, more rapidly, with greater duration and/or with greater affinity with one or more particular target(s) than with alternative substances (e.g., other targets or non-target species). For example, a capture molecule may “bind” to a target if it attaches to the target with greater affinity, avidity, more readily, and/or with greater duration than it attaches to other substances. In at least one example, the capture molecule may be an antibody that specifically or at least preferentially binds to a target (e.g., an antigen specific to the antibody) with greater affinity, avidity, more readily, and/or with greater duration than the antibody binds to other substances (e.g., non-target peptides or proteins). A capture molecule that specifically or preferentially binds to a first target may or may not specifically or preferentially bind to a second target. In some aspects, for example, a capture molecule may bind to two or more targets, wherein the nature of the binding with each target may be about the same or may be different (e.g., the capture molecule having greater affinity for one target as compared to another target). As such, “binding” does not necessarily require (although it can include) exclusive binding. In some aspects, reference to “binding” may refer to preferential binding, e.g., a preference for reaction or association with one or more targets as compared to other species or substances. The concept of “binding” also is understood to include the concept of specificity, e.g., selective attachment between two species (e.g., a capture molecule and a target). Specific binding may be biochemically characterized as saturable (non-specific binding being non-saturable). Further, according to some aspects of the present disclosure, targets may compete to bind to one or more specific binding sites of a capture molecule. Competitive binding may be demonstrated biochemically.

The capture molecule(s) may include, for example, one or more antibodies, peptides, proteins, or a combination thereof. Exemplary capture molecules suitable for the present disclosure include, but are not limited to, RNA, DNA, peptides, antibodies, aptamers, and protein-based aptamers. In at least one embodiment, the capture molecule is an antibody. In some aspects of the present disclosure, capture molecules may be blocked using blocking agents such as, e.g., serum, serum diluted in phosphate buffered saline (PBS), and other blocking agents known in the art.

Capture molecules according to the present disclosure may comprise, e.g., monoclonal antibodies, polyclonal antibodies, antibody fragments (e.g., Fab, Fab′, F(ab′)₂ , Fv, Fe, etc.), chimeric antibodies, single chain variable fragments (scFvs), mutants thereof, fusion proteins comprising an antibody portion, and/or any other polypeptide that comprises an antigen recognition site of the required specificity (including, e.g., antibody mimetics).

An “antibody” generally refers to an immunoglobulin molecule capable of specific binding to at least one target (e.g., an antigen specific to the antibody). Antibodies may have at least one antigen-recognition or antigen-binding site, which may be located in a variable region of the antibody. Antibodies according to the present disclosure may be capable of specific binding to a target such as a carbohydrate, polynucleotide, lipid, polypeptide, or other target through at least one antigen-recognition site of the antibody. The antibodies may be murine, rat, rabbit, chicken, human, or of any other origin, including humanized antibodies. Capture molecules, such as antibodies, may be made recombinantly and expressed using any suitable method. Further, in some aspects, the antibodies may be made recombinantly by phage display technology. Examples of expression and production methods may be found in U.S. Pat. Nos. 5,565,332; 5,580,717; 5,733,743 and 6,265,150; and Winter et al., Ann. Rev. Immunol., 12:433-455 (1994). The present disclosure is not limited to any particular source of antibody or manner in which the antibody is made. For example, antibodies may be made by hybridoma, phage selection, recombinant expression, transgenic animals, or other methods.

As used herein, the term “antibody” encompasses not only intact polyclonal and monoclonal antibodies, but also fragments thereof (e.g., Fab, Fab′, F(ab′)₂, Fv), single chain variable fragments (ScFv), mutants thereof, fusion proteins comprising an antibody portion, and any other modified configuration of the immunoglobulin molecule that comprises an antigen recognition site of the required specificity. The antibody may be an antibody of any class, including, but not limited to, IgG, IgA, or IgM (including any sub-class thereof), and antibodies not of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. Each class of immunoglobulin may have a distinct subunit structure and three-dimensional configuration.

The term “Fv” generally refers to an antibody fragment that comprises a complete antigen recognition and binding site. In a two-chain Fv species, this region may include a dimer of one heavy chain variable domain (VH) and one light chain variable domain (VL) in tight, non-covalent association. In a single-chain Fv species, one heavy and one light chain variable domain may be covalently linked by a flexible polypeptide linker, such that the light and heavy chains may associate in a dimeric structure analogous to the structure of a two-chain Fv species. In this configuration, three complementarity determining regions (CDRs) of each variable domain may interact to provide for antigen-binding specificity on the surface of the VH-VL dimer. In some aspects, however, a single variable domain (or half of a Fv comprising only three CDRs specific for an antigen) may have the ability to recognize and bind an antigen, although the single variable domain may have a lower affinity toward the antigen as compared to the affinity of the entire binding site.

A “monoclonal antibody” generally refers to a homogeneous antibody population, the monoclonal antibody comprising amino acids involved in the selective binding of an antigen. Monoclonal antibodies suitable for the present disclosure include naturally occurring and non-naturally occurring amino acids. A population of monoclonal antibodies may be at least partially specific, or highly specific, in the sense that they are generally directed to a single antigenic site, as opposed to polyclonal antibodies. The monoclonal antibodies encompassed herein includes intact monoclonal antibodies and full-length monoclonal antibodies, as well as fragments thereof (e.g., Fab, Fab′, F(ab′)₂, Fv), single chain (ScFv), mutants thereof, fusion proteins comprising an antibody portion, and any other modified configuration of the immunoglobulin molecule that comprises an antigen recognition site of the specificity and the ability to bind to an antigen (see discussion of antibodies above).

According to some aspects of the present disclosure, a capture molecule may bind a peptide epitope of two or more consecutive (i.e., sequential) amino acids. The amino acid(s) forming the target epitope may be linear or branched, and may comprise one or more amino acids that have been modified naturally or by intervention. For example, the amino acids may be modified via disulfide bond formation, glycosylation, lipidation, acetylation, phosphorylation, or any other manipulation or modification, such as conjugation with a labeling component. In some aspects, the amino acid(s) forming the target epitope may comprise one or more analogs of an amino acid (including, for example, unnatural amino acids) as well as other modifications. In some aspects, the target is a protein biomarker described herein.

In some embodiments, the capture molecule may bind its cognate target epitope with an affinity of binding reaction of at least about 10⁻⁷ M, at least 10⁻⁸ M, or at least about 10⁻⁹ M, or tighter binding. In some embodiments, a binding interaction may discriminate over adventitious binding interactions in the reaction by at least two-fold, at least five-fold, or a range of at least 10-fold to at least 100-fold or more.

In some aspects of the present disclosure, one or more capture molecules may be attached to, e.g., immobilized on, a surface. As used herein, the term “immobilized” includes being immobilized, bound, and/or linked to a surface, such as, e.g., a microbead. In at least some examples, the capture molecules may be attached to, or immobilized on, microbead surfaces. A microbead may be a particle having a generally curved shape. In at least one example, the microbeads may be spherical with a uniform diameter. Microbeads according to the present disclosure may be rigid, and may have a surface that is smooth or porous, or that includes both smooth portions and porous portions. A microbead may comprise one material or a combination of materials. The microbeads may have magnetic properties in some embodiments, e.g., the microbeads comprising a magnetic material or combination of materials.

According to some aspects of the present disclosure, the microbeads may have an average diameter between about 10 nm and about 100 μm, such as from about 50 nm to about 50 μm, from about 100 nm to about 10 μm, from about 100 nm to about 5 μm, from about 500 nm to about 5 μm, from about 100 nm to about 1 μm, from about 1 μm to about 50 μm, from about 5 μm to about 10 μm, or from about 10 μm to about 50 μm. For example, the microbeads may have an average diameter of about 10 nm, about 100 nm, about 500 nm, about 1 μm, about 5 μm, about 10 μm, about 50 μm, or about 100 μm.

Linking of a capture molecule to a surface may be covalent or non-covalent. Linking capture molecules to the microbeads may be achieved by any suitable method(s). For example, the surfaces of the microbeads may be functionalized with one or more chemical functional groups, e.g., to be conjugated to capture molecules. Exemplary functional groups include, but are not limited to, amine, thiol, phosphate, alkyl, alkene, alkyne, arene, alcohol, ketone, aldehyde, carboxyl, and alkoxy groups. In at least one example, the microbeads may be conjugated to antibodies, or any other capture entity as described below.

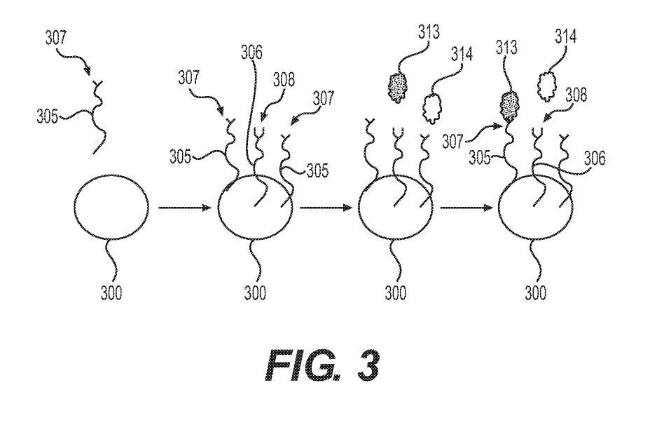

FIG. 3 is a schematic showing a microbead 300 for use in some aspects of the present disclosure. As shown, two different types of capture molecules 305, 306 may be attached to the surface of the microbead 300. Each capture molecule 305, 306 may be covalently bonded to the surface via any suitable chemical linking group or entity of the capture molecule 305, 306 and/or of the surface of the microbead 300, such that a binding site 307, 308 of the respective capture molecules 305, 306 is available for binding to a target. When combined with a sample comprising a plurality of biomarkers 313, 314, capture molecule 305 may selectively bind to biomarker 313, but not to biomarker 314. Further, capture molecule 306 may not be specific to biomarker 314, such that it does not bind to biomarker 314. Thus, biomarker 313 may be detected via its association with the microbead and capture molecule 305, whereas biomarker 314 may not be detected.

While FIG. 3 illustrates an example wherein different types of capture molecules are attached to a single microbead (e.g., for capture and detection of different targets), a plurality or set of microbeads may include only one type of capture molecule, such that the microbeads are specific to one target. For example, one or more capture molecules specific to a particular biomarker may be attached to each microbead of a plurality of microbeads. Each microbead of the plurality of microbeads may have the same size, shape, and chemical composition as the other microbeads, or the plurality of microbeads may include at least one microbead having a different size, shape, and/or chemical composition than at least one other microbeads of the plurality of microbeads.

In some examples, the capture molecule(s) may be labeled, e.g., comprising at least one detectable label (e.g., a chemical tag or probe molecule). For example, the capture molecule(s) may comprise a label detectable by an analytical technique such as optical detection, e.g., fluorescence, chemiluminescence, or electrochemiluminscence. In some aspects, the capture molecule(s) may comprise a fluorescently-labeled antibody or fluorescently-labeled protein.

Some aspects of the present disclosure include analyzing a sample in a diagnostic assay to determine the presence or absence of one or more biomarkers, and/or measuring the amount of one or more biomarkers in the sample. In at least one embodiment, the assay may comprise one or more capture molecules. For example, the assay may comprise a plurality or set of capture molecules. In some embodiments, the set may comprise at least two distinct capture molecules, wherein each distinct capture molecule may recognize a different target (e.g., a biomarker, such as a peptide). The set of capture molecules may range from 2 to 80 capture molecules. In some embodiments, the set of capture molecules may comprise at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or 80 or more distinct capture molecules. In at last one example, the set of capture molecules includes 5, 6, or 7 distinct capture molecules each specific to a different biomarker related to a particular health condition, e.g. cancer. In another example, the set of capture molecules includes 12 distinct capture molecules each specific to a different biomarker related to a particular health condition. In yet another example, the set of capture molecules includes between 20 and 60 distinct capture molecules each specific to a different biomarker related to a particular health condition. In some examples, one or more other target binding agents may be used, in addition to the capture molecules and capture molecule sets described herein.

The choice of capture molecules may depend on the desired application and/or the biomarkers to be detected in the sample. The capture molecule(s) may be specific to one or more biomarkers of a set of biomarkers, e.g., a biomarker panel. As discussed above, capture molecules may include antibodies specific to biomarkers associated with a particular health condition. In some aspects, each capture molecule may be specific to one biomarker of the panel.

Exemplary panels may comprise from 1 to 80 biomarkers, e.g., from 1 to 60 biomarkers, from 1 to 40 biomarkers, from 1 to 30 biomarkers, from 1 to 20 biomarkers, from 1 to 15 biomarkers, from 1 to 12 biomarkers, from 2 to 60 biomarkers, from 2 to 15 biomarkers, from 2 to 12 biomarkers, from 3 to 60 biomarkers, from 3 to 20 biomarkers, from 3 to 12 biomarkers, from 4 to 60 biomarkers, from 4 to 12 biomarkers, from 4 to 8 biomarkers, from 5 to 60 biomarkers, from 5 to 40 biomarkers, from 5 to 20 biomarkers, from 5 to 12 biomarkers, from 5 to 7 biomarkers, from 6 to 20 biomarkers, or from 6 to 12 biomarkers, although panels with more than 20 biomarkers are also encompassed herein. For example, the panel may include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or 80 or more biomarkers.

All or a subset of the biomarkers of a panel may be associated with a disease or other health condition. For example, one or more biomarkers of a panel may be associated with a disease such as, e.g., cancer (including breast cancer, prostate cancer, or ovarian cancer), a respiratory disease, and/or heart disease. Biomarkers associated with respiratory diseases may include, e.g., biomarkers associated with influenza A, influenza B, anthrax, plague, and/or various allergens. In at last one example, the biomarker panel includes 5, 6, or 7 biomarkers related to a particular health condition, e.g., cancer.

In some embodiments, the biomarkers to be detected may be associated with breast cancer. In some exemplary methods of the present disclosure, the biomarkers for a breast cancer panel may include, but are not limited to, Her-2, MMP-2, CA 15-3, VEGF, OPN, p53, CA 125, CEA, and/or SER. For example, a biomarker panel useful for diagnostic information on breast cancer may include at least one biomarker chosen from Her-2, MMP-2, CA 15-3, VEGF, OPN, p53, CA 125, CEA, or SER. In some aspects, the biomarker panel may include each of Her-2, MMP-2, CA 15-3, VEGF, OPN, p53, CA 125, CEA, and SER. According to some aspects of the present disclosure, the biomarker panel may comprise at least two biomarkers chosen from CA 15-3, OPN, Her-2, MMP-2, and VEGF. For example, the biomarker panel may comprise CA 15-3, OPN, Her-2, MMP-2, and VEGF. Examples of sequence identifiers in the HUGO Gene Nomenclature Committee on-line database for such markers include Her-2 (X03363), MMP-2 (NM_004530), CA 15-3 (NM_002456), VEGF (MGC70609), OPN (NM_001040058), p53 (NM_000546), CA 125 (Q8WX17), and SER (NP 000116.2).

According to some aspects of the present disclosure, the capture molecules may comprise antibodies specific for breast cancer biomarkers. In some embodiments, the capture molecules may be specific for Her-2, MMP-2, CA 15-3, VEGF, OPN, p53, CA 125, and SER. In some embodiments, the capture molecules may be specific for Her-2, MMP-2, CA 15-3, and OPN. Exemplary capture antibodies specific for breast cancer markers may include, but are not limited to: (1) anti-Her-2 (e.g., monoclonal anti-human ErbB2 antibody, Catalog #MAB1129, Clone #191924, supplied by R&D systems), (2) anti-matrix metallopeptidase 2 (MMP-2) (e.g., monoclonal anti-human MMP-2 antibody, Catalog #MAB902, Clone #36006.211, supplied by R&D systems), (3) anti-CA 15-3 (e.g., monoclonal anti-human CA 15-3 antibody, Catalog #10-0O3, Clone #M8071022, supplied by Fitzgerald Industries), (4) anti-osteopontin (OPN) (e.g., monoclonal anti-human osteopontin antibody, Catalog #MAB1433, Clone #190312 supplied by R&D Systems), and (5) anti-vascular endothelial growth factor (VEGF) (e.g., VEGF purified mouse anti-human, Catalog #AHGO114, Clone #A183C 13G8, supplied by ThermoFisher Scientific). In some embodiments, the capture molecules may be Clone 191924, Clone 36006.211, Clone M8071022, and Clone 190312.

Exemplary biomarkers for a prostate cancer panel (e.g., biomarkers useful in obtaining diagnostic information regarding prostate cancer) may include, but are not limited to, PSA. Exemplary biomarkers for an ovarian cancer panel (e.g., biomarkers useful in obtaining diagnostic information regarding ovarian cancer) may include, but are not limited to, CA 125. Exemplary biomarkers for a heart disease panel (e.g., biomarkers useful in obtaining diagnostic information regarding heart disease) may include, but are not limited to, troponin T, troponin I, CRP, homocysteine, myoglobin, and/or creatine kinase. The capture molecule(s) used for detection of the above biomarker panels may be specific to one or more biomarkers of the panel. For example, the capture molecules may comprise antibodies specific to the biomarkers associated with each respective disease or health condition.

The number of capture molecules in a set of capture molecules may depend on one or more of the following parameters: the contemplated uses and applications of the set of capture molecules, the complexity of the sample, the average size of the proteins in the sample, the frequency that the cognate target epitope is present or predicted to be present in a sample, the binding affinity and/or specificity of the capture molecules, knowledge of the target protein(s), and/or the stability of the capture molecules.

According to some aspects of the present disclosure, “detect” may refer to identifying the presence, absence and/or amount of a target or other species to be detected. Detection may be done visually and/or using any suitable device, such as, e.g., a scanner and/or detector. Further, any suitable analytical technique may be used for detection, including, but not limited to, optical techniques. Non-limiting examples of techniques that may be used in detection according to the present disclosure include absorbance, fluorescence, chemiluminescence, and electrochemiluminescence.

The detection of a target (e.g., a biomarker) bound to a capture molecule may be made using a detectable label. For example a detection molecule specific to a target may bind to the target (e.g., the target also being bound to a capture molecule) for detection of the target. In some examples, the detection molecules may comprise detection antibodies. Such detection antibodies may be specific for a biomarker target, e.g., similar to the specificity of some capture antibodies to a biomarker target as discussed above. Exemplary detection molecules suitable for the present disclosure may include, but are not limited to, (1) anti-Her-2 (e.g., polyclonal goat anti-human ErbB2 antibody, Catalog #AF-1129, supplied by R&D Systems),(2) anti-MMP-2 (e.g., polyclonal goat anti-human MMP-2 antibody, Catalog # AF-902, supplied by R&D Systems), (3) anti-CA 15-3 (e.g., monoclonal anti-human CA 15-3 antibody, Catalog #10-0O3B, Clone #M8071021, supplied by Fitzgerald Industries), (4) anti-osteopontin (e.g., polyclonal goat anti-human osteopontin antibody, Catalog #AF-1433, R&D Systems), and (5) anti-VEGF (e.g., polyclonal rabbit anti-human VEGF biotin conjugated antibody, Catalog #AHG9119, supplied by ThermoFisher Scientific).

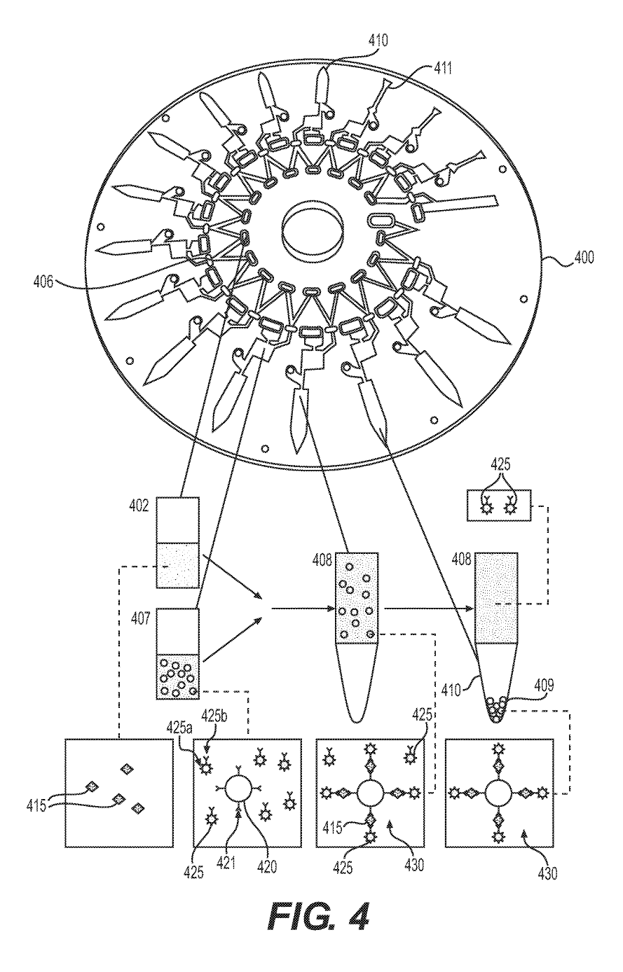

Detection molecules may be labeled using any suitable method(s). In some examples, the detection molecules may comprise at least one detectable label (e.g., a chemical tag or probe molecule) that is detectable by an analytical technique such as optical detection, e.g., absorbance, fluorescence, chemiluminescence, or electrochemiluminescence. For example, the detectable label may comprise fluorescent agents, colorimetric agents, magnetic agents, or electrical agents, or any combination thereof. Fluorescent agents include, but are not limited to, quantum dots and fluorophores, e.g., including Alexa Fluor® 546 dye molecules and Alexa Fluor® 488 dye molecules produced by ThermoFisher Scientific, phycoerythrin (PE), and allophycynin (APC). In some aspects of the present disclosure, a detection molecule may include one or more detectable labels before binding to a target (see, e.g., FIG. 4 below). In other aspects, one or more detectable labels may be added after a detection molecule is bound to the target. For example, after binding a target to a capture molecule (e.g., the capture molecule being attached to a microbead) and to a detection molecule to form a capture molecule/target/detection molecule complex, the complex may be mixed with one or more detectable labels, such that the detectable labels react and attach to the complex. Addition of the detectable label(s) may be performed in a separate reaction chamber. For example, after the capture molecule/target/detection molecule complex is formed, the complex may be separated from any unbound/unreacted reagents via a density medium prior to reaction with the detectable label(s).

In some aspects of the present disclosure, the assay may include competitive binding. For example, the capture molecule-microbead reagents pre-loaded into the microfluidic disc may include a detection molecule having a detectable label. When a sample comprising a target is mixed with the reagents, the target may compete with the detection molecule to bind to the capture molecule-microbead. The amount of reagents may be chosen such that, in the absence of any targets, a high signal is detected, providing a reference signal. A decrease in the amount of signal relative to the reference signal may indicate the presence of targets (e.g., having displaced the detection molecules in binding to the capture molecule-microbeads). The decrease in signal relative to the reference signal may be used to determine the concentration of the target. This type of assay may be useful for targets of limited size or number of epitopes, including, e.g., small proteins with only one epitope.

Exemplary Devices

Devices suitable for various embodiments of the present disclosure may provide for point-of-care testing, e.g., to obtain diagnostic information for patient at or near the time and place of patient care. For example, the device may be portable and/or self-contained. Further, devices according to the present disclosure may be used to measure multiple targets (e.g., biomarkers) simultaneously, in a multiplex assay.

In some aspects, the device may include microfluidic channels for performing a multiplex assay. On a microfluidics platform, for example, a relatively small volume of sample (e.g., on the order of microliters (μL)) may be sufficient to measure levels for a plurality of biomarkers. For example, the device may be a microfluidic-based immunoassay detection device comprising a microfluidic disc, a motor to control the spinning rate of the disc, and a detector such as an optical reader, e.g., to measure biomarkers. In some embodiments, the microfluidic disc may contain microbeads conjugated with specific capture antibodies and a suitable set of reagents for binding, detection, and separation processes. Microfluidic devices according to the present disclosure may include any of the features disclosed in U.S. Provisional Application No. 62/202,353, which is incorporated by reference herein.

Microfluidic discs of the present disclosure may comprise one or more channels that include a series of interconnected chambers, wherein reagents and sample may be mixed and/or moved from chamber to chamber by applying a centrifugal force. Thus, for example, the microfluidic disc may provide the channel(s) through which fluid flows and the chambers where reagents are stored and/or mixed with a sample added to the disc in a diagnostic assay.

The channel or channels of the microfluidic disc may be any suitable shape including, e.g., round, trapezoidal, triangular, or other geometric shapes. Channels may be straight, curved, zig-zag, U-shaped, or other configurations, e.g., depending upon the application and function of the channel. Channel sizes may be selected based on one or more factors, such as the type(s) and/or number of targets (e.g., biomarkers) to be analyzed in a sample, the type(s) and/or number of capture molecules stored in the disc for binding with the target(s), the nature of binding between targets and capture molecules, among other factors. In some exemplary discs, the channels may be from about 0.01 microns to 5 millimeters deep and from 0.01 microns to about 5 millimeters wide. For example, the channels may range from about 0.05 microns to about 5 millimeters deep and from about 0.01 microns to about 1 centimeter or more in diameter. The fluid capacity of the channels may range from about 1 nanoliter to about 1 mL or more, depending upon the application.

The microfluidic discs may be made of any material or combination of materials suitable for the assay. For example, the microfluidic disc may comprise one or more polymers or copolymers. Exemplary materials suitable for the microfluidic discs herein include, but are not limited to, polypropylene, polystyrene, polyethylene, acrylates such as poly(methyl methacrylate) (PMMA), cyclic olefin polymers (COP), cyclic olefin copolymers (COP), polydimethylsiloxane (PDMS), polyacrylamides, and combinations thereof.

FIG. 1 shows an exemplary microfluidic disc 100 comprising one microfluidic channel that includes a series of interconnected chambers through which fluid may flow during an assay. The number and design of the chambers may be tailored to the particular targets being detected and reagents used. As shown, for example, the channel may include a sample inlet 102, a sample preparation chamber 104, a metering and reaction chamber 106, a separation chamber 108, and a detection chamber 110. The disc 100 may include a central aperture 105, e.g., for coupling the disc 100 to a powered component to drive rotation of the disc 100 during an assay. In an exemplary procedure, a sample, e.g., a blood sample that includes the biomarkers of interest, is added to the sample inlet 102 of the microfluidic disc 100. In general, an aliquot of raw sample (e.g., whole blood or other biological fluid) ranging from about 1 μL to about 300 μL or more (˜one to several drops) may be added to the inlet, such as from about 1 μL to about 280 μL, from about 1 μL to about 250 μL, from about 1 μL to about 220 μL, from about 1 μL to about 200 μL, from about 1 μL to about 180 μL, from about 1 μL to about 150 μL, from about 1 μL to about 120 μL, from about 1 μL to about 100 μL, from about 1 μL to about 80 μL, 1 μL to about 80 μL, from about 1 μL to about 40 μL, from about 1 μL to about 20 μL, from about 1 μL to about 6 μL, from about 20 μL to about 250 μL, from about 20 μL to about 200 μL, from about 50 μL to about 100 μL, from about 50 μL to about 250 μL, from about 100 μL to about 200 μL, from about 5 μL to about 80 μL, or from about 2 μL to about 5 μL. For example, an aliquot of sample of about 1 μL, about 2 μL, about 3 μL, about 4 μL, about 5 μL, about 6 μL about 20 μL, about 40 μL, about 60 μL, about 80 μL, about 100 μL, about 120 μL, about 150 μL, about 180 μL, about 200 μL, about 220 μL, about 240 μL, about 250 μL, about 280 μL, or about 300 μL may be used. As the disc rotates, the sample may flow through the channel, radially outward.

In some embodiments, microfluidic discs may include a valving system with relatively narrow channels, e.g., to regulate fluid flow. For example, the microfluidic disc 100 of FIG. 1 may include a valve 111 between the sample preparation chamber 104 and the metering and reaction chamber 106 and/or between the metering and reaction chamber 106 and the separation chamber 108. Valves may provide resistance to fluid flow through the channels until enough force is provided to overcome such resistance. An example of force to overcome such resistance may include centrifugal force applied by spinning the disc at threshold speed. Each valve may be designed or adjusted to correspond to a particular rotational speed or speeds, e.g., such that different chambers may be selectively accessed to move the fluid at a desired time according to the operations of the device.

The sample preparation chamber 104 may provide for any pre-processing of the sample prior to mixing with reagents stored in the disc 100. For example, various components of the sample may be separated, e.g., via a filter, such that only a portion of the original sample may flow through the channel for analysis. For example, the sample inlet 102 may be configured to separate whole blood into plasma, serum, and cell components. In some examples, the amount of sample component (e.g., blood plasma) mixed with reagents for analysis may generally range from about 1 μL to about 6 μL. For example, the amount of sample or sample component sufficient for a multiplex assay according to the present disclosure may range from about 2 μL to about 5 μL, e.g., an aliquot of sample of about 1 μL, about 2 μL, about 3 μL, about 4 μL, about 5 μL, or about 6 μL. Excess sample and/or any components of a raw sample not used for analysis may be separated into a waste chamber, e.g., in communication with the metering and reaction chamber 106.

The metering and reaction chamber 106 may combine the sample with the capture molecules, e.g., for binding targets to the capture molecules, and determine the appropriate volume for subsequent steps of the assay. Thus, for example, reagents comprising capture molecules attached to microbeads (see, e.g., FIG. 3) may be pre-loaded and stored in the disc prior to an assay. In some examples, the disc 100 may comprise a metering chamber for measuring out the appropriate volume of sample for analysis that is separate from the reaction chamber, where the reagents may be stored for mixing with the sample as the sample enters the reaction chamber. Reagents other than the capture molecule-microbeads may be present in liquid, gel or lyophilized form, such that the capture molecule/microbeads are suspended in the liquid, gel, or lyophilized material(s). When a portion of the reagents are lyophilized, the sample or sample component introduced into the metering and reaction chamber 106 (e.g., blood plasma) for analysis may reconstitute the lyophilized material(s).

The separation chamber 108 and the detection chamber 110 (which may comprise the end of the microfluidic channel) may provide for collection of the bound capture molecule-microbead/target complex for detection of the target. For example, the channels may comprise a density medium, e.g., having a density less than that of the microbeads and greater than that of unbound reagents, such as Ficoll. After reaction with the sample, the microbeads may be moved through the density medium in the separation chamber 108 to separate them from other reagents and to allow the collection of the beads in a pellet in the detection chamber 110. The pellet then may be analyzed by a detector to determine and analyze the presence and/or concentration of targets.

FIG. 2 shows an exemplary disc 200 comprising a plurality of microfluidic channels according to some aspects of the present disclosure. Each channel may include, or be in communication with, at least one sample inlet 202, at least one sample preparation chamber 204, at least one metering chamber 206, at least one reaction chamber 207, at least one separation chamber 208, and at least one detection chamber 210. For example, the channels may extend radially outward at regularly spaced intervals. In some aspects, the number of separation chambers 208 and detection chambers 210 (for detection of a target) may be greater than the number of sample inlets 202. As shown, for example, the disc 200 includes 12 sample inlets 202 each leading into a sample preparation chamber 204. Each of the 12 sample preparation chambers 204 is in communication with 5 metering chambers 206. Each metering chamber 206 leads into a reaction chamber 207 (e.g., where reagents may be pre-loaded into the disc 200), a separation chamber 208, and a detection chamber 210. Thus, the disc 200 may have a total of 60 channels, providing for analysis of 12 different samples, and 5 different biomarkers per sample (e.g., if each reaction chamber 207 includes reagents specific to a different biomarker). Each channel may include one or more valves similar to valve 111 of FIG. 1. The microfluidic disc 200 may include a central aperture 205 similar to aperture 105 of disc 100 in FIG. 1.

FIG. 4 shows a schematic of an exemplary assay using a microfluidic disc 400 according to some aspects of the present disclosure, which may include any of the features of microfluidic discs 100 and/or 200 discussed above. The disc 400 may include a plurality of channels, each channel including a metering chamber 406, and a reaction chamber 407, a separation chamber 408, and a detection chamber 410. Upon rotation of the disc 400, fluid may flow through the channels, radially outward. As shown, two sample inlets 402 may be divided between two channels, e.g., at chamber 406. Thus, an aliquot of a fluid sample (e.g., whole blood) added to each sample inlet 402 may be divided between two channels. The sizes and configurations of the channels may permit a specified volume of sample to proceed through the channel for mixing with the reagents. In some aspects, for example, chamber 406 may serve to meter an appropriate amount of sample to each reaction chamber 407. While not shown in FIG. 4, the sample inlets 402 may lead into sample preparation chambers as discussed above, e.g., to separate various components of the sample prior to metering an appropriate volume of sample to be mixed with reagents for analysis.

Each reaction chamber 407 may include reagents pre-loaded in the microfluidic disc 400. The reagents may include a plurality of microbeads 420 with capture molecules 421 covalently attached to the surface, such that a binding site at the free end of each capture molecule 421 is available for binding with a target of the sample. FIG. 4 shows an exemplary microbead 420 with four capture molecules 421 attached to the surface, however more or fewer than four capture molecules 421 per microbead 420 may be used. The reagents also may include a plurality of detection molecules 425, each detection molecule including a binding site 425a for binding to a target, and a detection label 425b such as a fluorescence agent. The capture molecules 421 and the detection molecules 425 may be specific to the same target(s) to be detected.

In some aspects, each reaction chamber 407 may include capture molecules 421 and/or detection molecules 425 specific to the same target (s) as the other reaction chambers 407 of the disc 400. In other examples, at least one of the channels may include capture molecules 421 and/or detection molecules 425 specific to different target(s) as compared to at least another one of the channels of the disc 400. In yet other examples, each channel of the microfluidic disc 400 may include different capture molecules 421 and/or detection molecules 425, such that different targets may be captured and detected in each channel or each separation chamber 408. Further, the disc 400 may include capture molecules 421 and/or detection molecules 425 pre-loaded in the same reaction chamber 407 and specific to different targets. For example, a single reaction chamber 407 may include a first plurality of capture molecules 421 (e.g., attached to microbeads 420) specific to a first target and a plurality of capture molecules 421 (e.g., attached to microbeads 420) specific to a second target different from the first target.

The sample introduced in the sample inlets 402 may include one or more targets, e.g., biomarker 415. For example, the biomarkers 415 to be detected in the sample may be antibodies, and the capture molecules 421 may be capture antibodies. Thus, for example, each capture molecule 421 may bind to a biomarker 415, such that each microbead 420 binds four biomarkers 415, as shown schematically in FIG. 4. Further, each detection molecule 425 may bind to one of the biomarkers 415 attached to the microbeads 420, thus forming a detection complex 430.

The detection complexes 430 may flow to the separation chamber 408. In some aspects, a density medium as discussed above may be used to separate the detection complexes 430 from any unbound sample and/or reagents (e.g., unbound detection molecules 425) in the separation chamber 408, as shown schematically in FIG. 4 (illustration of separation chamber 408, left). Upon application of sufficient centrifugal force due to rotation of the disc 400, the detection completes 430 may move from the separation chamber 408 to the detection chamber 410 to collect together in a pellet 409 at the base of the detection chamber 410, also shown schematically in FIG. 4 (illustration of separation chamber 408, right). The separation chambers 408 and/or detection chambers 410 of the disc 400 may have the same shape, or different shapes. For example, the as shown the disc 400 includes 13 channels with detection chambers 410 having a generally tapered, V-shaped base, and 3 channels with detection chambers 411 having a flat-shaped base.

When the detection method is chemiluminescence or electrochemiluminscence, the disc 400 may include a suitable substrate/reagent pre-loaded into the detection chambers 410 and/or 411, such that the capture molecule-microbead/target complex may react with the substrate/reagent to generate light (e.g., ultraviolet, visible, or infrared light) for detection. In some aspects, the substrate/reagent may be present in a separate reservoir chamber, and may added to the pellet 409 in the same chamber where the pellet was generated (e.g., detection chamber 410) or in a separate chamber where the pellet 409 and the substrate/reagent are combined.

As mentioned above, some aspects of the present disclosure include biomarkers associated with a particular disease or other health condition. For example, a microfluidic disc (which may be substantially similar to disc 400 of FIG. 4) may include a set of capture molecules specific for a panel of biomarkers associated with breast cancer. The capture molecules may be immobilized on microbeads that are stored in the microfluidic disc. The biomarkers may comprise two or more biomarkers chosen from Her-2, MMP-2, CA 15-3, OPN, p53, VEGF, CA 125, CEA, and SER. For example, the device may include capture molecules specific for CA-15-3 and OPN, or capture molecules specific for Her-2, MMP-2, CA 15-3, and OPN.

Thus, for example, the plurality of capture molecules may include a plurality of capture antibodies specific to biomarkers associated with breast cancer. In some aspects, the plurality of capture antibodies may include at least two of the following capture antibodies: (1) anti-Her-2 (e.g., monoclonal anti-human ErbB2 antibody, Catalog #MAB1129, Clone #191924, supplied by R&D systems), (2) anti-matrix metallopeptidase 2 (MMP-2) (e.g., monoclonal anti-human MMP-2 antibody, Catalog #MAB902, Clone #36006.211, supplied by R&D systems), (3) anti-CA 15-3 (e.g., monoclonal anti-human CA 15-3 antibody, Catalog #10-0O3, Clone #M8071022, supplied by Fitzgerald Industries), (4) anti-osteopontin (OPN) (e.g., monoclonal anti-human osteopontin antibody, Catalog # MAB1433, Clone #190312 supplied by R&D Systems), and (5) anti-vascular endothelial growth factor (VEGF) (e.g., VEGF purified mouse anti-human, Catalog #AHGO114, Clone #A183C 13G8, supplied by ThermoFisher Scientific). In at least one example, the set of capture molecules includes (1) anti-Her-2 (e.g., monoclonal anti-human ErbB2 antibody, Catalog #MAB1129, Clone #191924, supplied by R&D systems), (2) anti-matrix metallopeptidase 2 (MMP-2) (e.g., monoclonal anti-human MMP-2 antibody, Catalog #MAB902, Clone #36006.211, supplied by R&D systems), (3) anti-CA 15-3 (e.g., monoclonal anti-human CA 15-3 antibody, Catalog #10-0O3, Clone #M8071022, supplied by Fitzgerald Industries), and (4) anti-osteopontin (OPN) (e.g., monoclonal anti-human osteopontin antibody, Catalog #MAB1433, Clone #190312 supplied by R&D Systems). Each channel may correspond to testing of a single biomarker (e.g., including capture molecules specific to a single biomarker) or multiple biomarkers (e.g., including capture molecules specific to two or more biomarkers, or a plurality of capture molecules each specific to a different biomarker).

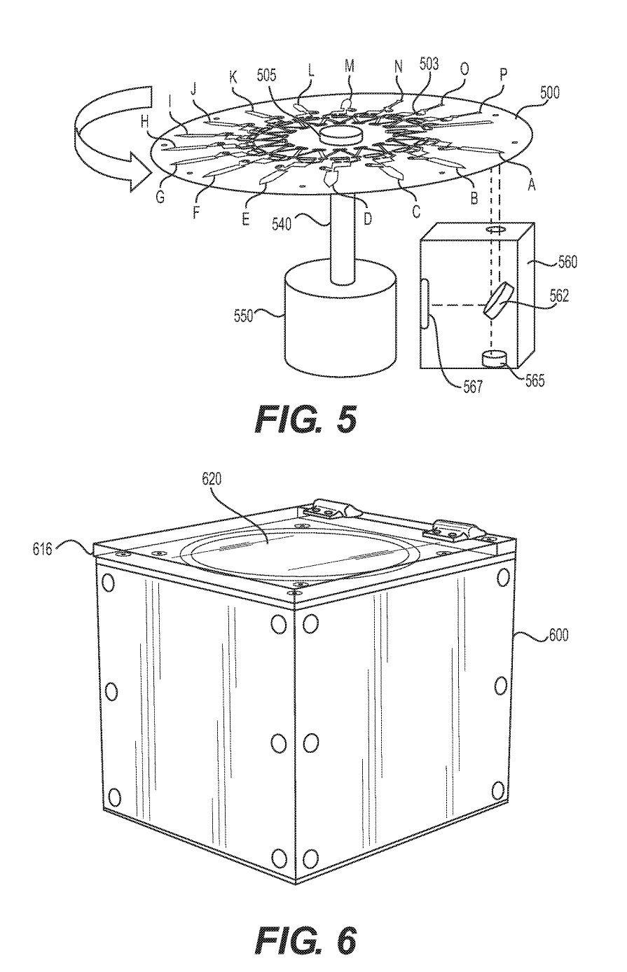

Devices according to the present disclosure may include a detection component for detecting a target (e.g., biomarker) bound to microbeads via capture molecules as discussed above. FIG. 5 shows an exemplary device comprising a microfluidic disc 500, a power source such as a motor 550, and a detection component 560. The disc 500 may include any of the features of discs 100, 200, and/or 400 discussed above, including, e.g., a plurality of channels 503 and a central aperture 505. The channels 503 may be in communication with a plurality of detection chambers, labeled sequentially A-P, as shown. The disc may be operably coupled to the motor 550 via a shaft 540, such that the motor 550 may power rotation of the disc 500 via the shaft 540. The motor may control rotation of the disc 500 counterclockwise (in the direction of the arrow shown in FIG. 5) and/or clockwise at a predetermined speed or series of predetermined speeds.

The detection component 560 may be configured to detect the presence of targets by measuring signals from detection molecules bound to the targets and collected in respective detection chambers A-P at or proximate the edge of the disc 500. For example, the detection component 560 may detect absorbance, fluorescence, chemiluminescence, or electrochemiluminscence, or any other type of signals from a detectable label of the disc 500. The amount of a target in each detection chamber A-P (and thus the concentration of the target in the original sample) may be determined based on the signal detected, the location of each detection chamber A-P relative to the others, and the rotation characteristics of the disc 500. For example, if the collection of signal begins when the detection component 560 is aligned with detection chamber A, as the disc 500 rotates, the amount of signal emitted from detection chamber A may be distinguished from the amount of signal emitted from detection chambers B-P based on the speed of rotation and the location of detection chamber A. Thus, for each full rotation of the disc 500, the detection component 560 may collect signal for each of detection chambers A-P. When the detection chambers A-P contain different targets (e.g., due to the use of different capture molecules to bind to the targets as discussed above), the concentrations of multiple targets present in the sample may be determined simultaneously or substantially simultaneously.

In some aspects, the detection component 560 may be an optical detector including a light source 565 for generating light, a detector 567, and optics 562 (e.g., mirrors and/or lenses) directing light from the light source 565 to the disc 500 and redirecting light emitted from the disc 500 to the detector 567. In at least one embodiment the detection component comprises light excitation at various wavelengths in the visible region and also outside the visible region, including, but not limited to a laser excitation, or a LED excitation and a CMOS sensor for detection of specific wavelengths, with the use of one or more appropriate filters and/or dichroic beam-splitters.

The detection component 560 may further include a reader for analyzing data from the detector 567 and a screen for displaying output from the reader. The reader may be optical. In some embodiments, the detection component 560 may include an imaging system, e.g., comprising a charge coupled device (CCD) camera. Output from the imaging system may be displayed on a computer screen or other user interface or viewing apparatus, including, but not limited to, e.g., a liquid crystal display (LCD) device. In some aspects, output from the imaging system may be transferred to a remote user interface such as a tablet computer or other computer controlled device such as a laptop or smartphone. The data may be transferred via wire or wireless communication, including, but not limited to, Bluetooth, and/or may be stored or archived on remote servers, e.g., in the Internet cloud.

FIG. 6 shows an exemplary housing 600 of a device according to some aspects of the present disclosure. For example, the housing may contain the device of FIG. 5. In some aspects, the housing 600 may include a cover (e.g., movable via hinges as shown or other suitable mechanism) and a door 620 that may be opened and closed for inserting and removing a microfluidic disc.

Analyzing a sample according to the present disclosure may include determining a value or a set of values associated with a given sample by one or more quantitative and/or qualitative measurements. For example, “analyzing” according to some embodiments of the present disclosure includes measuring constituent expression levels in a sample obtained from a subject and comparing the levels against constituent levels in a sample or set of samples from the same subject (e.g., the samples being collected at different times to assess the progression of a potential health condition) or other subject(s) (e.g., for comparison to a confirmed medical diagnosis of disease or lack of disease in another subject). The data obtained according to the present disclosure may include the presence or absence of specific biomarker or biomarkers in the sample or the presence or absence of the plurality of biomarkers in the sample. In some embodiments, the data may include the concentration of a plurality of biomarkers in a sample, and their relative presence compared to a physiological level, e.g., to determine over-expression, normal-expression, or under-expression of a plurality of biomarkers.

In at least one embodiment, scoring the sample comprises analyzing the data and outputting a score. A “score” may include, but is not limited to, a value or set of values that may be selected and/or used for analytic, comparison, diagnostic, and/or other purposes according to the present disclosure. In some embodiments, for example, a score may be used to assess a subject's health condition or medical condition based on, e.g., a measured amount of one or more constituents (e.g., targets, such as biomarkers) of a sample obtained from the subject.

Analysis of the data can include use of a predictive model. A “predictive model” may include, but is not limited to, a mathematical construct developed using an algorithm or combination of algorithms for grouping sets of data, e.g., to allow for discrimination of the grouped data. A predictive model according to the present disclosure may be developed using any suitable mathematical and/or statistical methods including, but not limited to, principal component analysis (PCA) and/or linear discriminant analysis (LDA). In some examples, the grouped data includes data for each biomarker of a panel of biomarkers.

PCA is a technique that may be used to reduce multidimensional data sets to lower dimensions for analysis. Mathematically, PCA may be defined as an orthogonal linear transformation that transforms data to a new coordinate system, such that the greatest variance by any projection of the data comes to lie on the first coordinate (called the first principal component), the second greatest variance on the second coordinate, and so on. PCA may be used as a tool in exploratory data analysis and for making predictive models. PCA also may include calculation of the eigenvalue decomposition of a data covariance matrix or singular value decomposition of a data matrix, e.g., after mean centering the data for each attribute. The results of a PCA may be discussed in terms of component scores and loadings.

LDA is a method that may be used to find the linear combination of features that best separates two or more classes of objects or events. The resulting combination may be used as a linear classifier, or, alternatively, for dimensionality reduction before later classification.

The present disclosure includes a method for scoring a sample from a subject, the method comprising, e.g., categorizing a human sample using quantitative data associated with a plurality of biomarkers, wherein the biomarkers are associated with a particular disease or other health condition, e.g., breast cancer. For example, the method of categorizing the sample may use data associated with a plurality of biomarkers associated with breast cancer. In some aspects, the plurality of biomarkers associated with breast cancer includes at least CA 15-3 and OPN. Additional biomarkers may include, e.g., Her-2, MMP-2, VEGF, p53, CA 125, CEA, and/or SER. For example, the plurality of biomarkers may include CA 15-3, OPN, Her-2 and MMP-2, or CA 15-3, OPN, Her-2, p53, CA 125, CEA, and SER. In at least one embodiment, the method of categorizing a sample uses data associated with the following biomarkers: CA 15-3, OPN, Her-2, and MMP-2.

Analysis according to some methods of the present disclosure may include categorizing a sample (e.g., categorizing the levels of biomarkers of a sample according to a biomarker panel) into categories according to a score produced with the predictive model. Overexpression of biomarkers may generally be understood as a sign of disease. Based on the amount of overexpression, an appropriate score and category may be assigned. Disease categories and corresponding biomarker levels have been reported. See, for example, U.S. Application Publication No. 2008/0200342 A1, incorporated by reference herein. Categories may include, for example, a healthy categorization (e.g., disease-free), an early-stage disease categorization, and a late-stage disease categorization. For example, the categorization may be chosen from a healthy categorization, an early-stage disease categorization, or a late-stage disease categorization.

Diagnostic information obtained according to the present disclosure may be compared to reference data of biomarker levels measured for patients with a confirmed diagnosis of the same disease or health collection. The probability that the diagnosis is correct may be calculated, e.g., as a linear regression of the data to compute specificity and sensitivity of the panel. The probability that categorization is correct may be model-dependent and/or biomarker-dependent, and can be at least 60%, at least 70%, at least 80%, at least 87%, at least 90%, or at least 95% correct. In some embodiments, a probability that the categorization is correct may be at least 60%, at least 70%, at least 80%, at least 87%, at least 90%, or at least 95%. In one example, a single biomarker for the screening of patients with breast cancer may provide a predictive value less than 70%, whereas a panel of biomarkers tested simultaneously may provide a combined effect to increase the predictive value to greater than 90%, e.g., a predictive value of 91%.

As mentioned above, as a disease or other health condition evolves or progresses over time, some or all biomarkers of a biomarker panel may be overexpressed, and continue to be overexpressed. Thus, measuring the biomarkers at different times (e.g., over months and/or years, according to the stage or aggressiveness of the disease) may provide information regarding disease progression in the subject. For example, biomarker levels may be measured every 1, 2, 3, or 4 weeks, every 1, 2, 3, 4, 5, 6, 7, 8, 9, 10, 11, or 12 months, or every 1, 2, 3, 4 or 5 years. For each set of measurements of a biomarker panel, a score may be determined as discussed above. In some aspects, a first score (e.g., based on the measured levels of biomarkers in a biomarker panel at a first time) for a first sample obtained from a subject may be compared to a second score (e.g., based on the measured levels for the same biomarker panel at a second, later time) determined for a second sample obtained from the subject. This comparison also may be used e.g., to determine the progress or effectiveness of therapy for the treatment of disease. A difference between the first score and the second score may indicate a disease stage, such as a disease stage of breast cancer. In some embodiments, the score may be used to diagnose a neoplastic breast disease, such as breast cancer.

The following examples are intended to illustrate the present disclosure without, however, being limiting in nature. It is understood that the present disclosure encompasses additional embodiments consistent with the foregoing description and following examples.

### EXAMPLES

### Example 1

A multiplex assay for a panel of 5 biomarkers of breast cancer is performed using a microfluidic disc as follows:

(1) Whole blood sample is obtained from a subject from a finger prick or venipuncture.

(2) A drop of the blood is introduced in an inlet chamber of the disc via a metered disposable transfer pipette.

(3) The disc is placed into a microfluidics device that contains a motor as a power source and an optical detector, and the cover of the device is closed. The disc is positioned such that it is coupled to the motor (which controls the direction and speed of rotation of the disc) and positioned above the detector, the detector being located towards the periphery of the disc. The device includes a program for running a breast cancer screening assay by rotation of the disc at a series of predetermined speeds as described in steps (4)-(11) below. The assay program is activated.

(4) The disc is spun at 1000 revolutions per minute (rpm) to transfer the sample from the inlet chamber to a blood separation chamber.

(5) The disc is then spun at 3000 rpm to separate blood cells from plasma in the blood.

(6) The disc is then spun at 250 rpm to siphon the plasma into individual metering chambers (5 μL of plasma into each metering chamber) in communication with the blood separation chamber.

(7) The disc is then spun at 750 rpm to eliminate extra plasma into a waste chamber in communication with the metering chambers.

(8) The disc is then spun at 1000 rpm to move the plasma from the metering chambers to respective incubation chambers in communication with the metering chambers. Each incubation chamber contains reagents (capture antibodies attached to microbeads, detection antibodies, and any additives such as salts and/or buffers to control pH) specific to one of the biomarkers of the panel for assaying the biomarker. The reagents include the microbeads (attached to the capture molecules) suspended in liquid, gel, or lyophilized material(s).

(9) The disc is spun at 300 rpm in a single direction or in alternating clockwise and counterclockwise directions, to improve reconstitution of reagents and the kinetics of reaction between the biomarkers and the reagents.

(10) The disc is then spun at 2000 rpm to move the incubated sample into separation chambers in communication with the respective incubation chambers, where the biomarkers bound to microbeads form pellets within respective detection chambers at the periphery of the disc.

(11) While the disc is kept spinning at 2000 rpm, detection of the biomarkers of the pellets is performed by the optical detector positioned under the plane of the disc in proximity of the detection chambers.

(12) The raw signal (fluorescence, luminescence, and/or absorption) is recorded, amplified and analyzed. The signal is recorded and is correlated to each biomarker to determine the concentration of each biomarker in the original blood sample.

### Example 2

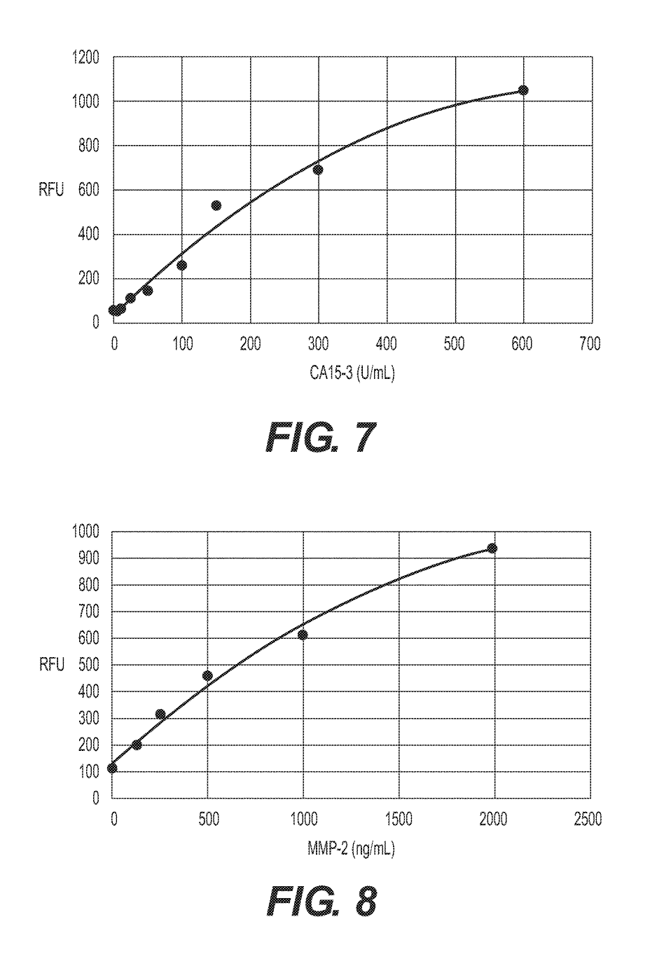

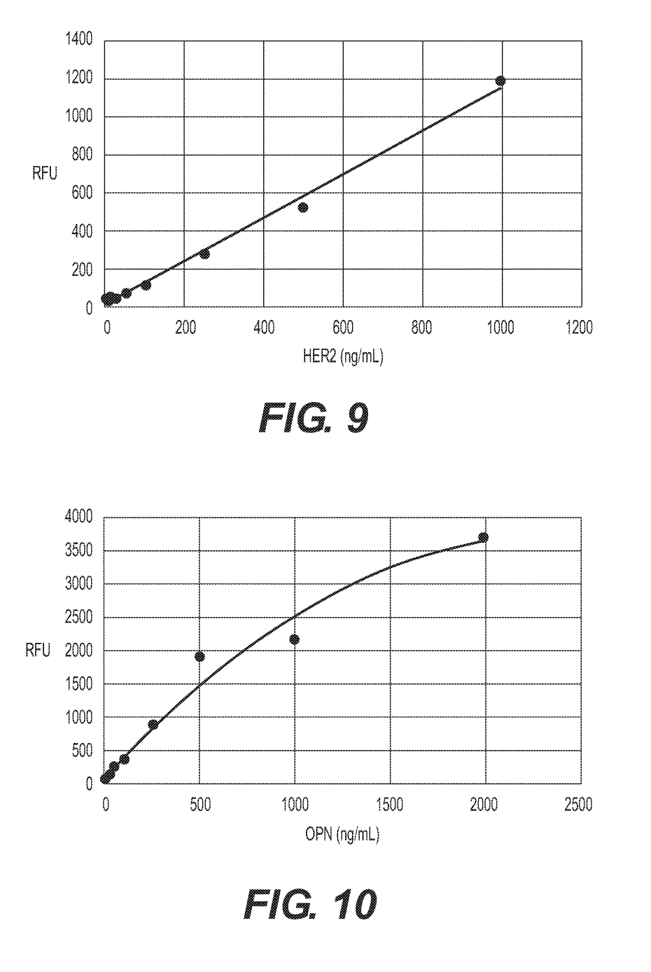

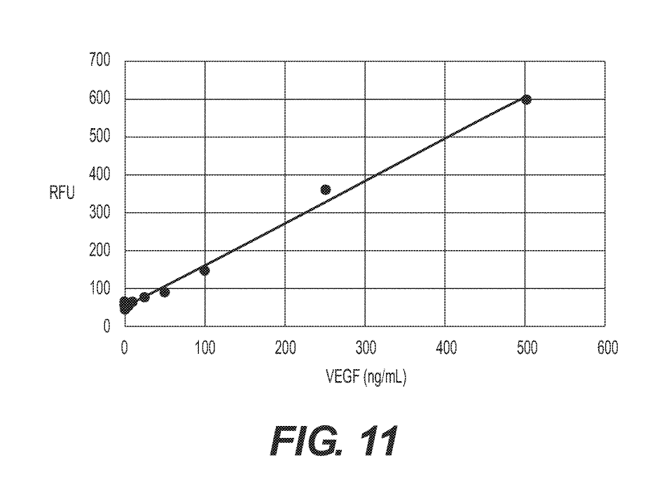

Microfluidic discs in accordance with the present disclosure was used to measure fluorescence for various concentrations of the following biomarkers of breast cancer: CA 15-3, MMP-2, Her-2, OPN, and VEGF. The steps of the assay were substantially similar to those of Example 1. Fluorescence was measured in relative fluorescence units (RFU). Results are shown in FIGS. 7-11. Each disc included 12 inlets, each inlet in communication with 5 metering chambers, and each metering chamber in communication with a reaction chamber, a separation chamber, and a detection chamber (total of 60 channels). An aliquot of 5 μL of human plasma was used for each sample with known concentrations of the respective biomarkers spiked in. One disc was used for each biomarker, with different concentrations of the biomarker being added to different inlets. The incubation time for the reaction in each case was 15 minutes.

The following capture antibodies and detection antibodies were used, wherein the capture antibodies were immobilized on 1 μm diameter silica beads functionalized with carboxyl groups, and the detection antibodies were conjugated with phycoerythrin (PE):
CA 15-3: Capture antibody=anti-CA 15-3; detection antibody=anti-CA 15-3.
MMP-2: Capture antibody=anti-MMP-2; detection antibody=anti-MMP-2.
Her-2: Capture antibody=anti-Her-2; detection antibody=anti-Her-2.
OPN: Capture antibody=anti-OPN; detection antibody=anti-OPN.
VEGF: Capture molecule=anti-VEGF; detection antibody=anti-VEGF.

The sample was moved from the sample inlet to the other chambers/compartments of the microfluidic circuitry by centrifugal forces, with the final step of creating a pellet of the beads conjugated with the capture antibody, within the detection chamber, at the edge of the disc, where the total fluorescence of the pellet was recorded.

It is intended that the specification and examples be considered as exemplary only, with a true scope and spirit of the present disclosure being indicated by the following claims.

What is claimed is:

1. A device comprising:
a disc including a plurality of microfluidic channels extending in a radial direction of the disc, the microfluidic channels comprising a plurality of capture molecules specific to at least one biomarker, wherein each capture molecule of the plurality of capture molecules is attached to a particle.

2. The device of claim 1, wherein the plurality of microfluidic channels comprises a first channel including a plurality of first capture molecules specific to a first biomarker and a second channel including a plurality of second capture molecules specific to a second biomarker different from the first biomarker.

3. The device of claim 2, wherein the disc includes from 12 to 60 microfluidic channels.

4. The device of claim 1, wherein each capture molecule is attached to a microbead having an average diameter ranging from 100 nm to 10 μm.

5. The device of claim 4, wherein each capture molecule is attached to a microbead having an average diameter of about 1 μm.

6. The device of claim 1, wherein each capture molecule of the plurality of capture molecules comprises an antibody.

7. The device of claim 1, wherein the plurality of capture molecules includes capture molecules specific for biomarkers chosen from human estrogen receptor 2 (Her-2), matrix metallopeptidase-2 (MMP-2), cancer antigen 15-3 (CA 15-3), vascular endothelial growth factor (VEGF), tumor protein p53 (p53), cancer antigen 125 (CA 125), carcinoembryonic antigen (CEA), or serum estrogen receptor (SER) or osteopontin (OPN).

8. The device of claim 1, wherein the plurality of capture molecules includes at least one antibody chosen from antibodies Clone 191924, Clone 36006.211, Clone M8071022, or Clone 190312.

9. The device of claim 1, further comprising a power source and a detector, wherein the detector is configured to detect fluorescence or chemiluminescence.

10. The device of claim 1, wherein the disc comprises at least one sample inlet configured to separate plasma from whole blood.

11. (canceled)

12. A method of detection of biomarkers using the device, comprising:
introducing a fluid sample into at least one microfluidic channel of the disc;
rotating the disc, such that the fluid sample flows radially outward through the at least one microfluidic channel to combine with at least one capture molecule of the plurality of capture molecules; and
detecting a signal from the disc indicative of a presence of at least one biomarker of the fluid sample.

13. The method of claim 12, wherein the fluid sample comprises a plurality of biomarkers associated with a health condition.

14. The method of claim 13, wherein the health condition is breast cancer.

15. The method of as in claims 12, wherein the fluid sample comprises at least one biomarker chosen from human estrogen receptor 2 (Her-2), matrix metallopeptidase-2 (MMP-2), cancer antigen 15-3 (CA 15-3), vascular endothelial growth factor (VEGF), osteopontin (OPN), tumor protein p53 (p53), cancer antigen 125 (CA 125), carcinoembryonic antigen (CEA), or serum estrogen receptor (SER).

16. The method of as in claim 12, wherein the plurality of capture molecules includes capture molecules specific for biomarkers associated with breast cancer.

17. The method of as in claim 12, wherein the plurality of capture molecules includes capture molecules specific for at least one biomarker chosen from human estrogen receptor 2 (Her-2), matrix metallopeptidase-2 (MMP-2), cancer antigen 15-3 (CA 15-3), vascular endothelial growth factor (VEGF), osteopontin (OPN), tumor protein p53 (p53), cancer antigen 125 (CA 125), carcinoembryonic antigen (CEA), or serum estrogen receptor (SER).
